(19) United States
(12) Patent Application Publication
(10) Pub. No.: US 2019/0076213 A1
(43) Pub. Date: Mar. 14, 2019

(54) DENTAL DEVICE WITH FOOT PEDAL CONTROLLER

(71) Applicant: DENTSPLY SIRONA Inc., York, PA (US)

(72) Inventor: Jay A. FAYER, Crystal Lake, IL (US)

(21) Appl. No.: 16/130,013

(22) Filed: Sep. 13, 2018

Related U.S. Application Data
(60) Provisional application No. 62/557,835, filed on Sep. 13, 2017.

(51) Int. Cl. A61C 1/00

(52) U.S. Cl. CPC A61C 1/0023; A61B 2017/00977

(57) ABSTRACT

A dental device is disclosed comprising a dental handpiece, a controller including a microcontroller and a foot control pressure sensor, and a foot pedal. The foot pedal is in communication with the controller and controls the dental handpiece. The dental device is arranged to configure between at least two of a speed limiting mode, a feathering mode, and a standard mode. In the speed limiting mode, the controller proportions air flow so as to limit the speed of the dental handpiece to a specified speed. In the feathering mode, the foot pedal proportions air to the controller which then transmits the air to the dental handpiece such that the foot pedal controls the speed of the dental handpiece. In the standard mode, the speed of the dental handpiece is set without limiting by the controller and without feathering by the foot pedal.

### CROSS REFERENCE TO RELATED APPLICATIONS

This application relates to and claims the benefit of U.S. Provisional Patent Application No. 62/557,835, filed Sep. 13, 2017, entitled “Foot Pedal and Dental Device,” the disclosures of which are incorporated by reference in their entirety.

### FIELD OF THE INVENTION

The present invention is directed to foot pedals useful for controlling a dental device, such as a low speed or high speed dental handpiece.

### BACKGROUND OF THE INVENTION

Foot pedal controls for dental tools such as handpieces are typically limited in the available modes of control offered, the level of fine control over rotational speed possible, and the ability to facilely and intuitively change modes of operation. There is a continuing need for improved control devices for dental tools such as handpieces.

### BRIEF DESCRIPTION OF THE INVENTION

In one embodiment, a dental device comprises a dental handpiece, a controller including a microcontroller and a foot control pressure sensor, and a foot pedal. The foot pedal is in communication with the controller and controls the dental handpiece. The dental device is arranged to configure between at least two of a speed limiting mode, a feathering mode, and a standard mode. In the speed limiting mode, the controller proportions air flow so as to limit the speed of the dental handpiece to a specified speed. In the feathering mode, the foot pedal proportions air to the controller which then transmits the air to the dental handpiece such that the foot pedal controls the speed of the dental handpiece. In the standard mode, the speed of the dental handpiece is set without limiting by the controller and without feathering by the foot pedal.

Other features and advantages of the present invention will be apparent from the following more detailed description of the preferred embodiment, taken in conjunction with the accompanying drawings, which illustrate, by way of example, the principles of the invention.

### BRIEF DESCRIPTION OF THE DRAWINGS

FIG. 1 is a schematic view of a dental device, according to an embodiment of the present disclosure.

FIG. 2 is a side view of a dental handpiece, according to an embodiment of the present disclosure.

FIG. 3 is a schematic representation of the connections of a dental device, according to an embodiment of the present disclosure.

Wherever possible, the same reference numbers will be used throughout the drawings to represent the same parts.

### DETAILED DESCRIPTION OF THE INVENTION

Provided is a dental device with a foot pedal controller. Embodiments of the present disclosure, in comparison to compositions of matter and methods not utilizing one or more features disclosed herein, include increased modes of operation of a dental device, increased degree of control of a dental device, more efficient mode selection of a dental device, or combinations thereof.

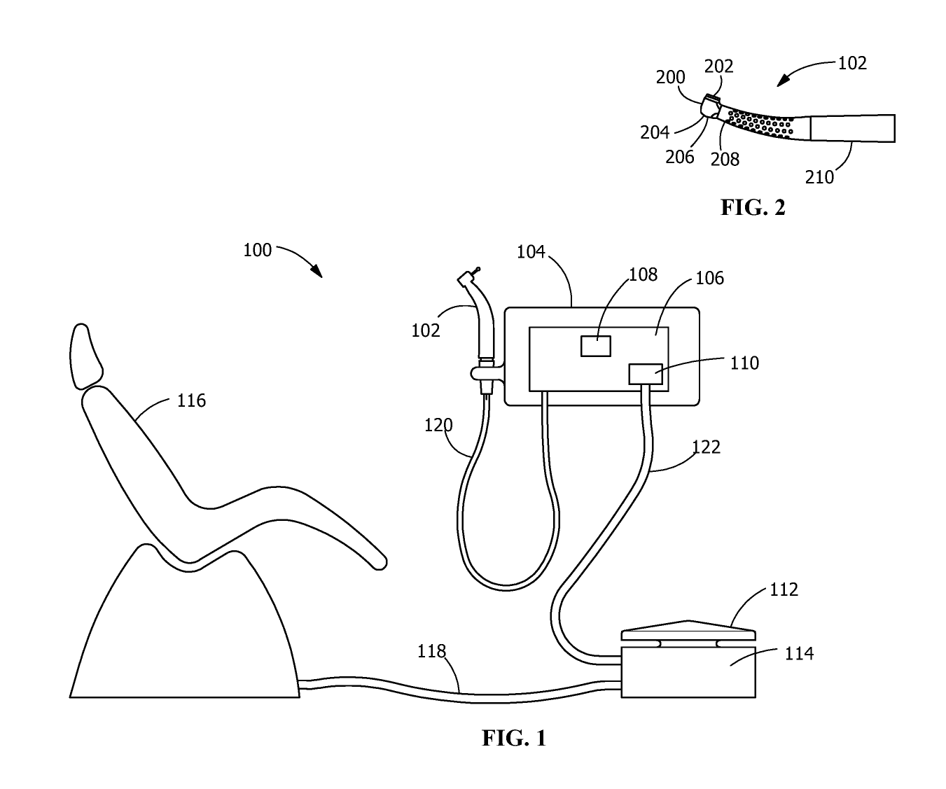

Referring to FIG. 1, in one embodiment a dental device 100 includes a dental handpiece 102, which may be used in a dentist's office. The dental device 100, which may be placed in a dental treatment center 104, may include a controller 106 which includes a microcontroller 108 and a foot control pressure sensor 110. The foot control pressure sensor 110 may be connected to a foot pedal 112, such as, but not limited to, a pneumatic foot control 114. The controller 106 may also be connected to the dental handpiece 102 and may thereby control the dental handpiece 102. By way of example, the controller 106 may control the speed of the dental handpiece 102 based on the information received from the dental handpiece 102. Although depicted in FIG. 1 as being present in a dental treatment center 104, the controller 106 may be located elsewhere, including, but not limited to, in a standalone device.

In one embodiment, a dental treatment chair 116 is connected to the pneumatic foot control 114 by a supply air hose 118, the pneumatic foot control 114 is connected to the dental handpiece 102 by a handpiece hose 120, and the pneumatic foot control 114 is connected to the controller 106 by a foot control hose 122.

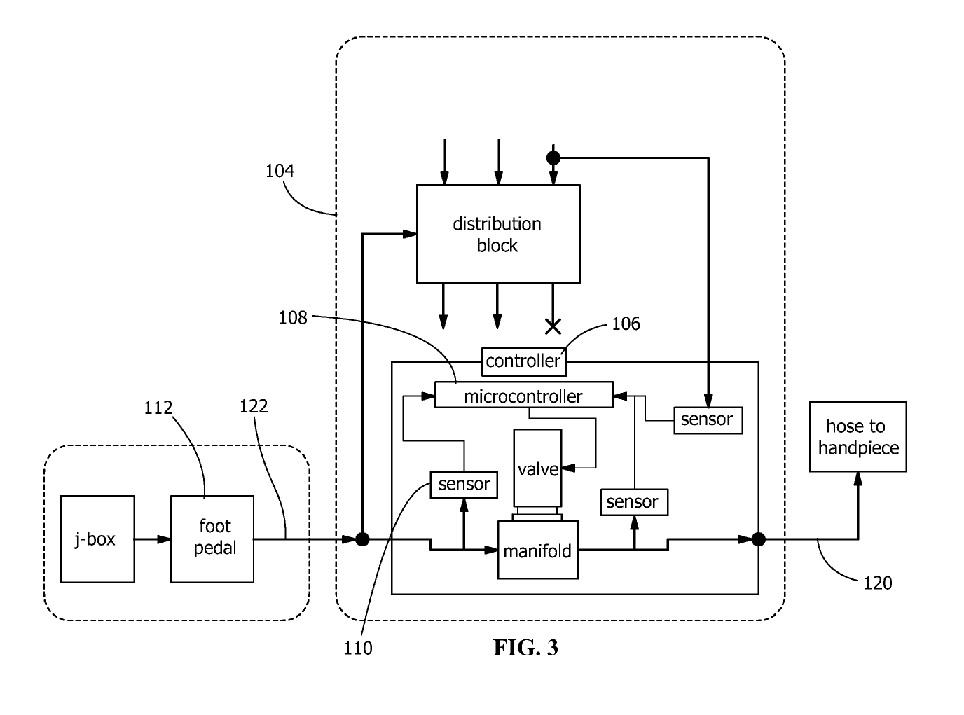

Referring to FIG. 2, the dental handpiece 102 may be suitable for use as a medical device. In one embodiment, the dental handpiece 102 includes a handpiece head 200, a push button 202, a cooling spray outlet 204, a chuck system opening 206, a light aperture 208, and a handpiece sleeve 210. The light aperture 208 may be configured to indicate different modes of use for the dental device 100 to a practitioner, or to indicate various self-test modes for the dental device 100.

Referring to FIG. 3, the controller 106 may include any suitable arrangement of sensors, valves, and manifolds, and fluid distribution blocks, working in conjunction with the microcontroller 106 to coordinate the foot control pressure sensor 110 with the dental handpiece 102.

Referring to FIGS. 1-3, the dental handpiece 102 may be a high-speed or low-speed handpiece. In one embodiment, the controller 106 monitors the foot pedal 112 for a pressure applied thereto, and the foot control pressure sensor 110 converts air pressure to a voltage which the microcontroller 108 reads directly with an internal analog-to-digital converter.

The foot pedal 112 may control the speed of the dental handpiece 102, signal a change in the mode of the dental handpiece 102, or both, by communicating with the controller 106.

In one embodiment, the foot pedal 112, which may also be identified as a rheostat, proportions air to the controller 106, which then transmits the air on to the dental handpiece 102 to drive a turbine, which then rotates a bur. In this way a practitioner may control the power delivered to the dental handpiece 102, and thereby control the speed of the dental handpiece 102. This mode of speed control is generally referred to as feathering and may be the typical means of dental handpiece 102 speed control by the practitioner.

The controller 106 may measure the speed of the dental handpiece 102. In one embodiment, the controller 106 proportions the air it receives from the foot pedal 112 before the air is delivered on to the dental handpiece 102. Thus, the controller 106 may determine speed and proportion air so as to limit the speed of the dental handpiece 102 to any specified speed. In one embodiment, the specified speed may be, but is not limited to, 330,000 rpm and under. This mode of operation may generally be referred to as the speed limiting mode.

The microcontroller 108 may continually compare the pressure from the foot pedal 112 with a fixed pressure level, above which the foot pedal 112 is considered pressed, and below which, the foot pedal 112 is considered released.

The microcontroller 108 may apply a time-stamp to each transition of the foot pedal 112, from released-to-pressed and/or from pressed-to-released. By comparing the current time with the most recent time-stamp, the microcontroller 108 may determine how long the foot pedal 112 has been pressed or released.

In one embodiment, the microcontroller 108 keeps a record of recent foot pedal 112 transitions and their time stamps, coded as a state number. From this record, if the microcontroller 108 determines that the foot pedal 112 is now just pressed, but previously had been pressed for less than 1 second and then released for less than 1 second, the speed control mode is switched to feathering mode. In a further embodiment, if the microcontroller 108 determines that the foot pedal 112 has been released for more than 0.9 seconds, the speed control mode returns to speed limiting mode. The sequences and time durations for these steps and mode adjustments may be adjusted as would be understood by a person having ordinary skill in the art.

In another embodiment, operation of the dental device 100 may be switched into a mode without the speed-limiting or pressure change features being active (direct foot pedal 112 control of the dental handpiece 102), by any suitable predetermined control motion, such as, but not limited to, double-tapping the foot pedal 112 (press-release-press). The dental handpiece 102 will then run in standard mode (high-speed without speed-limiting or low-speed without speed-limiting, depending on the dental device 100 configuration) and will remain in standard mode until the foot pedal 112 is released. A practitioner operating in standard mode must keep the foot pedal 112 depressed in order to run the dental handpiece 102 at high speed or low speed, as no air will pass to the dental handpiece 102 unless the foot pedal 112 is pressed.

Any mode change may be indicated to a practitioner by predetermined visual signaling of the light aperture 208. In one embodiment, the light aperture 208 is the intra-oral light found on many dental handpieces 102 and which are used to illuminate the oral cavity.

In one embodiment, the controller 106 may sense pressure from the foot pedal 112 and the controller 106 may determine if there was one short press of the foot pedal 112 just before it is pressed and held to run the dental handpiece 102. If there was one short press of the foot pedal 112, the controller 106 does not speed-limit but instead institutes a feathering mode, which sets a valve to a constant setting that sends to the dental handpiece 102 its recommended operating pressure when the foot pedal 112 is fully pressed, and then lets the foot pedal 112 do all the proportioning of air to the dental handpiece 102. As described above, this feathering mode is achieved because the foot pedal 112 proportions air to the controller 106, which then transmits the air on to the dental handpiece 102 to drive a turbine, which then rotates a bur. In this way a practitioner may control the power delivered to the dental handpiece 102, and thereby control its speed.

The foot pedal 102 may further engage in certain self-test features, such as, but not limited to, a dental handpiece 102 assessment. With the dental handpiece 102 in a holder, a practitioner may institute a self-test by performing a specific sequence of foot taps, pedal depressions, and/or movement with the dental handpiece 102. In one embodiment, the practitioner may institute a self-test by pressing and releasing the foot pedal 112 twice and then pressing the foot pedal 112 once more and holding it down, and then lifting the dental handpiece 102 from the holder. The dental handpiece 102 will run as the controller 106 performs a self-assessment of the dental handpiece 102 based on the pressure required to maintain free speed.

In another embodiment, three presses of the foot pedal 112, then holding down the foot pedal 112, institutes a self-test on the installation pressure, with results indicated on the light aperture 208 of the handpiece 102. In yet another embodiment, four presses of the foot pedal 112, and then holding down the foot pedal 112, institutes a calibration of a calibrate valve.

The foot pedal 112 may be utilized in a variety of ways, including various numbers and sequences of presses and holds to switch the dental handpiece 102 between various modes. As one of ordinary skill in the art will understand, the specific order of pedal presses and releases discussed herein are merely for guidance and the dental device 100 may be programmed to respond to any specific preordained movements.

While the invention has been described with reference to a preferred embodiment, it will be understood by those skilled in the art that various changes may be made and equivalents may be substituted for elements thereof without departing from the scope of the invention. In addition, many modifications may be made to adapt a particular situation or material to the teachings of the invention without departing from the essential scope thereof. Therefore, it is intended that the invention not be limited to the particular embodiment disclosed as the best mode contemplated for carrying out this invention, but that the invention will include all embodiments falling within the scope of the appended claims.

What is claimed is:

1. A dental device, comprising:
a dental handpiece;
a controller including:
a microcontroller; and
a foot control pressure sensor; and
a foot pedal,
wherein the foot pedal is in communication with the controller and controls the dental handpiece,
wherein the dental device is arranged to configure between at least two of:
a speed limiting mode in which the controller proportions air flow so as to limit the speed of the dental handpiece to a specified speed;
a feathering mode in which the foot pedal proportions air to the controller which then transmits the air to the dental handpiece such that the foot pedal controls the speed of the dental handpiece; and
a standard mode in which the speed of the dental handpiece is set without limiting by the controller and without feathering by the foot pedal.

2. The dental device of claim 1, wherein the dental device is placed in a dental treatment center.

3. The dental device of claim 1, wherein the dental device is placed in a standalone device remote from a dental treatment center.

4. The dental device of claim 1, wherein the foot pedal is a pneumatic foot control.

5. The dental device of claim 1, wherein the foot control pressure sensor converts air pressure to a voltage which the microcontroller reads directly with an internal analog-to-digital converter.

6. The dental device of claim 1, wherein the specified speed of the dental handpiece is 330,000 rpm.

7. The dental device of claim 1, wherein the microcontroller applies a time-stamp to each transition of the foot pedal from released-to-pressed and from pressed-to-released.

8. The dental device of claim 1, wherein a predetermined sequence of pressing and releasing the foot pedal switches the dental device from speed limiting mode to feathering mode.

9. The dental device of claim 1, wherein a predetermined sequence of pressing and releasing the foot pedal switches the dental device from standard mode to feathering mode.

10. The dental device of claim 1, wherein a predetermined sequence of pressing and releasing the foot pedal switches the dental device from speed limiting mode to standard mode.

11. The dental device of claim 1, wherein a predetermined sequence of pressing and releasing the foot pedal switches the dental device from feathering mode to standard mode.

12. The dental device of claim 1, wherein a predetermined sequence of pressing and releasing the foot pedal switches the dental device from standard mode to speed limiting mode.

13. The dental device of claim 1, wherein a predetermined sequence of pressing and releasing the foot pedal switches the dental device from feathering mode to speed limiting mode.

14. The dental device of claim 1, wherein a change of configuration between modes of the dental device is indicated by a predetermined visual signaling of a light aperture on the dental handpiece.

15. The dental device of claim 1, wherein a predetermined sequence of pressing and releasing the foot pedal institutes a self-test of the dental device.

16. The dental device of claim 15, wherein the self-test runs a self-assessment of the dental handpiece based on the pressure required to maintain free speed.

17. The dental device of claim 15, wherein the self-test runs a self-assessment of the installation pressure.

18. The dental device of claim 15, wherein the self-test runs a calibration of a calibrate valve.

19. The dental device of claim 15, wherein results of the self-test are indicated on the light aperture of the handpiece.

20. The dental device of claim 1, wherein the dental handpiece includes a handpiece head, a push button, a cooling spray outlet, a chuck system opening, a light aperture, and a handpiece sleeve.
